(19) United States
(12) Patent Application Publication
(10) Pub. No.: US 2006/0068011 A1
(43) Pub. Date: Mar. 30, 2006

(54) Simultaneous encapsulation technique for use in pharmaceutical compositions

(75) Inventor: Nkere Kanu Ebube, Round Lake, IL (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/955,045

(22) Filed: Sep. 30, 2004

(51) Int. Cl. A61K 9/24

(52) U.S. Cl. 424/472

(57) ABSTRACT

Multiple active pharmaceutical ingredients may be simultaneously encapsulated into pellets using the melt pelletization technique. The resulting pellets exhibit high active ingredient content and good handling characteristics, such as flowability, lubrication, compression, and size distribution, for production of a solid dosage form of a pharmaceutical composition. A method for preparing pellets is also disclosed.

### BACKGROUND OF THE INVENTION

The invention relates generally to a technique for the simultaneous encapsulation of multiple active ingredients into pellets for use in pharmaceutical compositions, and methods for use of the-pharmaceutical compositions. Multiple active ingredients may be simultaneously encapsulated into pellets using the melt pelletization technique resulting in uniform active ingredient distribution throughout the pellets for use in the preparation of solid dosage forms of pharmaceutical compositions, where the pellets exhibit desirable handling properties aiding in solid dosage form preparation. The melt pelletization technique is less expensive than other known techniques for the encapsulation of multiple active ingredients as used in solid dosage forms of pharmaceutical compositions.

### DESCRIPTION OF RELATED ART

Pharmaceutical compositions may be administered (or provided) to recipients in several forms, using different methods of delivery. For example, they may be provided orally, parenterally, intravascularly, intranasally, intrabronchially, transdermally, rectally, or vaginally to a recipient. Often, the preferable method of providing pharmaceutical compositions to a recipient is orally, either in a solid dosage form as a tablet or a capsule, or a liquid. Liquid pharmaceutical compositions may be difficult to administer due to the taste and viscosity of the solution, which can be unpleasant to the recipient. Further, liquid compositions often have limited shelf time, and must be timely prepared and presented to consumers. In addition, liquid pharmaceutical compositions must often be shipped and stored under controlled conditions, as extreme heat or cold may affect the effectiveness of the active ingredients.

For these and other reasons, many pharmaceutical compositions are prepared in a solid dosage form such as a tablet or a capsule. Methods for preparing solid dosage forms of pharmaceutical compositions are well known in the art. The active ingredients of the desired pharmaceutical composition may be mixed with inactive additives to form granules or pellets (also referred to as pelletized beads), which may then be compressed or otherwise formed and encapsulated into solid dosage forms as is well known to those skilled in the art. Many factors determine the composition of the mixture to be used to prepare the solid dosage form, as for example the strength of the final solid dosage form, the compressibility of the mixture, the absorption of the active ingredients within the recipient's body, the lubrication and flowability of the mixture for ease in manufacture of the solid dosage form.

Typically, the mixture includes inactive additives in addition to the active ingredients to impart desirable properties to the final product, or to aid in solid dosage formation. For example, many active ingredients are not readily compressible or may be hydrophobic or hydrophilic, causing problems during solid dosage formation. Alternatively, they may have a harsh taste, or may not readily disperse in the recipient's body upon ingestion. In these and other situations, inactive additives may be included in the mixture along with the active ingredients to aid in final solid dosage form preparation. Such inactive additives may include materials to act as binders, lubricants, diluents, disintegrating agents, coloring agents, flavoring agents, and preservatives.

The pharmaceutical composition typically is prepared in measured doses of active ingredients for provision to recipients. Generally, it is desirable for a recipient to receive the desired dosage of active ingredients in as few doses as possible. In addition, the physical size of a solid dosage form is limited to what can be comfortably and reasonably ingested by the intended recipient. Thus, where high amounts of inactive additives must be included in a mixture, the dosage of the active ingredients is necessarily reduced in each solid dosage form, leading to more doses required to obtain the desired pharmaceutical effect in the recipient.

In some situations, the mixture may be directly compressed to form the solid dosage form. Direct compression is limited in application due to poor flow characteristics and compressibility of the active ingredient(s), segregation and unacceptable content uniformity of the active(s). Inactive additives may be included in mixtures used in direct compression to improve compressibility and overall attributes of the dosage form.

Where direct compression is not feasible, common methods of preparing a mixture to be incorporated into a solid dosage form are dry granulation and wet granulation.

In dry granulation, the active ingredients are mixed optionally with inactive additives, screened, lubricated, and compressed to prepare the solid dosage form. For dry granulation to work, the mixture must have acceptable handling properties, such as flowability, lubrication, and compressibility for solid dosage formation.

Where the mixture does not exhibit acceptable handling properties, the process of wet granulation may be used. In wet granulation, binding agents (typically including a liquid) and optionally other inactive additives are mixed with the active ingredient(s), the wet mass is screened and dried, and the mixture is used to prepare the solid dosage form by, for example, compression or tableting.

Wet granulation is a common process used for mixture preparation for the manufacture of pharmaceutical compositions. U.S. Pat. No. 4,562,024, issued to Rogerson, the disclosure of which is hereby incorporated by reference in a manner consistent with this application, discloses a wet granulation process for preparing compressed tablets, where the active ingredient exhibits poor compressibility qualities. Part of a mixture of an active ingredient along with inactive additives are homogenized with granulating fluid to form a slurry, which slurry is used to moisten the remainder of the mixture. The mass thus formed is granulated and dried, and the granules are compressed and compacted into tablets.

Other disclosures describe using wet granulation methods to prepare sustained release pharmaceutical compositions. For example, U.S. Pat. Nos. 4,806,359; 4,820,522; 4,968,509; 5,004,613; 5,073,380; 5,200,193; and 5,462,747, each issued to Radebaugh et. al.; the disclosures of each which are hereby incorporated in their entireties in a manner consistent with this application, describe the formation of a sustained release pharmaceutical composition by making a wet granulation using povidone (PVP) in alcohol as the granulating fluid, mixing inactive ingredients such as erosion promoter, wicking agent, and lubricant with the wet mass, and compressing the resultant granulation.

U.S. Pat. No. 5,681,577, issued to Lech et. al., the disclosure of which is hereby incorporated by reference in a manner consistent with this disclosure, describes a multiple action cold/sinus preparation prepared by adsorbing the active ingredient mixture onto a silicon dioxide carrier in the wet granulation process. Such active ingredients as diphenhydramine, chlorpheniramine maleate, dextromethorphan hydrobromide, and an analgesic are contemplated for use in the preparation.

The wet granulation process is also described in U.S. Pat. Nos. 5,585,115; 5,725,883; 5,725,884; 6,103,219; and 6,217,909, each issued to Sherwood et. al., the disclosures of which are hereby incorporated by reference in their entireties in a manner consistent with this application. These disclosures describe additives to improve compressibility of wet granulation, which may also be added to dry granulation and direct compression mixtures. A slurry comprising a microcrystalline cellulose-based agglomerate with about 0.1% to about 20% silicon dioxide particles associated therein may be mixed with an active ingredient, wet granulated, and compressed into a solid dosage form.

Wet granulation is often a preferred method of preparing a mixture for manufacturing a solid dosage form of a pharmaceutical composition. However, other methods of preparing solid dosage forms are also known. For example, U.S. Pat. No. 5,662,935, issued to Motta, the disclosure of which is hereby incorporated in its entirety in a manner consistent with this application, describes exposing a mixture of active ingredients with one or more additives to mechanical or electromechanical ultrasonic energy for an established time and within a range of frequencies to provide a mixture for solid dosage formation.

U.S. Pat. No. 6,221,368, issued to Breitenbach et. al, the disclosure of which is hereby incorporated in its entirety in a manner consistent with this application, describes a process for producing solid dosage forms by mixing a polymeric binder and active ingredients and optionally other additives, and extruding the mixture to give the desired solid dosage form.

U.S. Pat. No. 6,194,005, issued to Farah et. al, the disclosure of which is hereby incorporated by reference in its entirety in a manner consistent with this disclosure, describes the preparation of a pharmaceutical composition exhibiting modified release of the active ingredient using a hot melt coating method. The mixture used to prepare the pharmaceutical composition is prepared by mixing a powder composed of an active ingredient and additives while heating to obtain grains, liquefying a lipid agent, coating the grains by spraying them with the liquefied lipid agent, and lowering the temperature of the product to cool the lipid agent to solidify around the grains.

Pelletizing is another method of preparing a mixture including an active pharmaceutical ingredient for preparing a solid dosage form. In the process of pelletization, powders or other granules are converted into essentially spherical beads, or pellets. In contrast to the particles formed by the process of granulation, pellets typically are more uniform in size distribution, tend to flow better, and can be more easily separated by size due to the more uniform shape of the pellets. In addition, pellets are known to provide a desirable surface morphology for further application of desired coatings or other additives.

U.S. Pat. No. 5,807,583, issued to Kristenson et. al., the disclosure of which is hereby incorporated by reference in its entirety in a manner consistent with this disclosure, describes a process for preparing sustained release pellets by pelletizing a mixture of active ingredient and a wax-like binder. The mixture is mechanically worked in a high shear mixer with sufficient mechanical energy input to melt the binder and pelletize the mixture, forming pellets having diameters ranging from about 0.2 mm to about 2.5 mm.

Similarly, U.S. Pat. No. 6,162,467, issued to Miller et. al., the disclosure of which is hereby incorporated by reference in a manner consistent with this application, describes a process for the manufacture of agglomerates. By working a mixture of an active ingredient and a carrier in a high speed mixer, breaking the agglomerates, and continuing the mixing with the optional addition of carrier or other additives, sustained release compositions can be formed by encapsulation of the agglomeration into capsules or tabletting, or by molding.

### SUMMARY OF THE INVENTION

It is an object of the invention to produce pellets including a mixture of multiple active pharmaceutical ingredients with optional inactive additives that can be formed into a solid dosage form.

It is also an object of the invention to produce pellets including a mixture of multiple active pharmaceutical ingredients with optional inactive additives that display a uniform distribution of the multiple active ingredients within each pellet that can be formed into a solid dosage form.

It is also an object of the invention to produce pellets of a mixture including multiple active pharmaceutical ingredients with optional inactive additives of a substantially uniform size that can be formed into a solid dosage form.

It is also an object of the invention to produce pelletized beads including a mixture of multiple active pharmaceutical ingredients with optional inactive additives having high concentrations of the active ingredients that can be formed into a solid dosage form.

It is further an object of the invention to produce pellets from a sparingly water-soluble or water-insoluble mixture including multiple active pharmaceutical ingredients with optional inactive additives that can be formed into a solid dosage form.

It is also an object of the invention to produce pellets including a mixture of multiple active pharmaceutical ingredients having optional inactive additives that exhibit desirable handling properties that can be formed into a solid dosage form.

Finally, it is an object of the invention to provide a method of preparing such pellets, and solid dosage forms of pharmaceutical compositions from such pellets.

In accordance with these and other features of the invention, there are provided pellets produced from a mixture including multiple active pharmaceutical ingredients with optional inactive additives produced by the melt pelletization process. According to one embodiment of the invention, the pellets have substantially uniform particle size distribution with a median particle size range of about 170 μm-245 μm. The pellets may be formed by subjecting a mix of multiple active pharmaceutical ingredients with at least one inactive additive, such as a binder, in a mixing device, imparting mechanical and thermal energy to the mixture until the binder has melted and pellets have formed. The pellets may then be cooled. In one embodiment of the invention, the pellets may be formed into a solid dosage form, such as for example a tablet or a capsule. The formation of solid dosage forms from pellets is well known to those skilled in the art, as described in U.S. Pat. No. 5,807,583 issued to Kristensen et. al., discussed infra. The pellets exhibit good uniformity of concentration of the multiple active pharmaceutical ingredients, substantially uniform particle size, good flowability, compression and lubrication, and other desirable handling characteristics for solid dosage formation.

In another embodiment of the invention, a more economical process than wet granulation for producing pellets including multiple active pharmaceutical ingredients is disclosed which produces a substantially uniformly sized pellet for the preparation of a solid dosage form of a pharmaceutical composition. The process comprises adding multiple active pharmaceutical ingredients along with at least one inactive additive, such as a binder, to a mixing device, adding thermal, and optionally mechanical energy, to the mixture, and mixing the multiple active pharmaceutical ingredients and binder until the pellets are formed, e.g., when the melting point of the binder is reached. The pellets formed by this process may then be cooled, and may be separated by size before being used to produce the solid dosage form.

In still another embodiment of the invention, pellets including multiple active pharmaceutical ingredients that are sparingly soluble or insoluble in water may be produced and subsequently used to prepare a solid dosage form.

In another embodiment, there is provided a method of preparing a solid dosage form of a pharmaceutical composition including multiple active pharmaceutical ingredients. The method comprises adding multiple active pharmaceutical ingredients to a mixing device; adding at least one inactive additive, such as a binder, to the mixing device; adding thermal energy to the mixing device; and mixing the multiple active ingredients and the binder until pellets are formed. A solid dosage form pharmaceutical composition is then formed from the pellets in any conventional manner known in the art.

In yet another embodiment of the invention, a process for treating cold and cough involves preparing a solid dosage form of a pharmaceutical composition from pellets including multiple active pharmaceutical ingredients and at least one optional inactive additive produced according to the invention, and administering the pharmaceutical composition to a recipient. In one embodiment, the pharmaceutical composition includes an expectorant, a cough suppressant and a nasal decongestant.

These and other features and advantages of the invention will be apparent to one skilled in the art upon reading the detailed description that follows.

### DETAILED DESCRIPTION OF THE INVENTION

The term “pharmaceutical composition” refers to a composition comprising one or more active ingredients that imparts a medicinal or therapeutic effect to, or is otherwise biologically active with relation to a recipient of the pharmaceutical composition. The recipient is preferably a human, but may be any living organism that exhibits such medicinal, therapeutic, or biological effects upon ingestion of the pharmaceutical composition.

The term “active ingredient” or “active pharmaceutical ingredient” refers to ingredients that cause or otherwise impart medicinal or therapeutic effects on the recipient of the ingredient upon ingestion, or that otherwise produce biological effects on the recipient. Typically, certain classes of active ingredients cause at least similar effects. For example, analgesics provide a pain-killing effect. The term “pharmaceutical active”, “active pharmaceutical agent”, “active agent” and “drug” as used herein should be considered to have the same meaning.

The term “Carr's Index” refers to a term used to assess the flow property and compressibility of bulk powders, and may be calculated by the following equation:
I=[1−V_{B}/V_{T}]×100
where I equals the Carr's Index. Powders with Carr's Index values of less than or equal to 25 are considered to have good flow characteristics. The Carr's Index of a mixture may be measured in accordance with the Test Methods detailed below.

The term “inactive additives” refers to ingredients included in pharmaceutical compositions that provide effects other than medicinal, therapeutic, or other biological effects to the recipient of the pharmaceutical composition. Examples of inactive additives are lubricants, diluents, compression agents, dispersion agents, disintegrating agents, coloring agents, flavoring agents, and preservatives. For example, compression agents may be necessary in a mixture to be formed into a solid dosage form to impart integrity and strength to the form, and to allow the solid dosage form to contain a sufficient amount of active ingredients in a fixed physical size. Although the compression agent may be beneficial to formation of the solid dosage form, it does not necessarily impart a medicinal, therapeutic, or other biological effect upon the recipient of the solid dosage form.

The term “solid dosage form” refers to a pharmaceutical composition in a solid form that is essentially ready for provision to a recipient. Although typically most solid dosage forms would be tablets, capsules, pills or other forms are also included. For example, a solid dosage form may be granules or powders intended for dispersion by the consumer into a liquid prior to ingestion. Other related dosage forms may include semi solids, suppositories, and are generally well known in the art.

In accordance with one embodiment of the invention, pellets used for preparing a solid dosage form of a pharmaceutical composition including multiple active ingredients may be prepared by mixing the active ingredients and optionally inactive additives such as a binder, placing the mixed ingredients in the bowl of a mixer that imparts sufficient mechanical and thermal energy to melt the binder and mixing the mixed ingredients until the melting point of the binder is reached and pellets are formed. Typically, the pellets containing the multiple active ingredients are cooled, either ambiently or in a controlled environment, before being manufactured into a solid dosage form. In a preferred embodiment, thermal energy may be added by circulating hot water through the jacket of a jacketed mixer bowl, although other forms of thermal energy may be used with either a jacketed or unjacketed mixer bowl, such as electrical, ultraviolet radiation, or microwave radiation. Further, mechanical energy preferably is input through the action of the impellers of the mixer, but other forms of mechanical energy may be used. The mixer may be high speed or high shear. The particle size of the pellets may be substantially uniform, but it may be desirable to further screen them after pelletizing to select an optimal size for further processing.

The amount of active ingredients in the mixture may vary, according to the desired composition of the solid dosage form. One advantage of the invention is that it allows for higher concentration of active ingredients in the solid dosage form due to higher active ingredient concentration in the pellets produced according to the methods and processes provided herein. The daily dosage may be included in a single delivery unit or may comprise multiple delivery units. Dividing the daily dosage among multiple delivery units may be desirable if a tablet is used, for example, to provide a tablet size that is convenient to swallow. If multiple delivery units are used, they may be administered at one time or administered at intervals during the dosage period (e.g. typically a day) if desired. Accordingly it should be understood that the amounts of the actives disclosed herein are for a dosage that may be delivered in a single delivery unit or multiple delivery units.

In a preferred embodiment of the invention, the multiple active ingredients in a pharmaceutical composition comprise components of cold and cough preparations, and may comprise an expectorant, a cough suppressant, and a nasal decongestant, and combinations thereof. A preferred expectorant is guaifenesin, although other expectorants may be used, such as potassium iodide, IPECAC, or sodium iodide. A preferred cough suppressant is dextromethorphan HBr, although other cough suppressants could be used such as benzonatate, codeine phosphate, carbetapentane tannage, or menthol. A preferred nasal decongestant is pseudoephedrine HCl, although other nasal decongestants could be used such as tetrahydrozoline HCL, oxymetazoline HCL, phenylephrine HCL or tannate, or zylometazoline HCL. Preferably, the weight ratio of the guaifenesin to dextromethorphan HBr to pseudoephedrine HCl should be about 85:5:10, on a dry weight basis. The content of each ingredient may vary by about 5 to about 10 % w/w and this would depend on the desired final pharmaceutical formulation. A recipient should preferably ingest about 200 to about 400 mg guaifenesin every four (4) hours per day, about 10 to about 20 mg dextromethorphan HBr every four (4) hours per day, and about 30 mg pseudoephedrine HCl every four (4) hours per day. The solid dosage form of the pharmaceutical composition may be designed to provide these preferred dosages in a minimum number of doses.

Other active ingredients may also be included, such as an analgesics or antihistamines. Preferred analgesics include acetaminophen, ibuprofen, aspirin, naproxin, ketoprofen, although any analgesic may be used. Preferred antihistamines include loratadine, fexofenadine, benadryl, other antihistamines which can be used are well known in the art.

Similarly, other types of formulation preparations may be prepared according to the invention. For example cold and sinus; cold and allergy; and cold, allergy and sinus. As used herein, the term “treat” or “treating” or “treatment” means to provide relief of one or more of the symptoms associated with cold, sinus, and allergy. Accordingly the pellets, and the method of preparing the pellets can be used in treating the patient of the related symptoms associated with the relative condition.

In a preferred embodiment, the multiple active ingredients may be mixed on an as-received basis, or may be pretreated before preparing the mixture of active ingredients and binder. The active ingredients can be coated or taste masked prior to pelletization. The active ingredients should be measured in the ratio they are desired in the pharmaceutical composition in solid dosage form, on a dry weight basis. For example, if the pharmaceutical composition having three active ingredients should have a ratio on a dry weight basis of 25/25/50 % w/w/w, the mixture prepared prior to pelletizing should contain a similar weight ratio on a dry weight basis. Once the dosage amount of each ingredient is determined per solid dosage form, those skilled in the art will be capable of calculating the amount of each active ingredient to add to the mix prior to pelletizing to achieve that final product content according to the disclosure provided herein without undue experimentation. The term “w/w”, unless otherwise indicated, means weight of a given component or specified combination of components to total weight of the composition expressed as a percentage.

The amount of binder added to the mixture will depend on the properties desired in the pellets, and may be limited by the amount of the active ingredients desired in the solid dosage form of the pharmaceutical composition. Since a single solid dose of the pharmaceutical composition is likely to be limited in size and therefore will contain a limited amount of each active ingredient, the amount of binder added to the mixture typically should be limited. However, the amount of binder must be sufficient to produce pellets that exhibit satisfactory qualities for solid dosage formation such as good flowability as measured by the Carr's Index (I), good particle size distribution, good lubrication, and good compressibility. A preferred value for I is less than or equal to 25, more preferably between 1 and 25, even more preferably between 1 and 15, and even more preferably between 1 and 10.

The binder used in the invention should have a low melting point relative to the active ingredients and can act as the adhesive for particle agglomeration and pellet formation. Preferred binders will exhibit the following properties: low melting point relative to the active ingredient; is inert; compatible with other ingredients; inexpensive; and safe. Two preferred binders are PEG 8000™, a polyethylene glycol available from Union Carbide Corporation of New Jersey and Gelucire 50/13™, available from Gattefossé S. A. of Westwood, New Jersey.

Other inactive additives may be included in the mixture as well to impart other desirable properties to the pellets. Such additives as lubricants, diluents, disintegrating agents, coloring agents, flavoring agents, preservatives or other excipients may be added. Those skilled in the art will be able to fashion a mixture for pelletizing including the types of additives desired in the pellets without undue experimentation, according to the disclosure provided herein.

The invention may be used to prepare any number of pharmaceutical compositions which use multiple active ingredients in a solid dosage form, such as a capsule or a tablet, and that exhibit the advantages described herein of substantially uniform particle sizes each having substantially uniform amounts of active ingredients, and that exhibit desirable handling and formulation characteristics such as good flowability, lubrication, and compressibility.

The invention is further illustrated by the following Examples, which should not be regarded as limiting.

### EXAMPLE 1

Pellets were prepared that simultaneously encapsulated guaifenesin, dextromethorphan HBr, and pseudoephedrine HCl. Guaifenesin is a white or slightly gray crystalline bitter-tasting powder, sparingly water soluble, with a molecular weight of 198.2. It is commonly used as an expectorant in doses ranging from 200 to 400 mg every four hours. Dextromethorphan HBr is a white, odorless crystalline powder with a molecular weight of 271.4, and is sparingly soluble in water. It is used as a cough suppressant, in doses ranging from about 10 mg to about 20 mg every four hours. Pseudoephedrine HCl is an odorous white to off-white crystal or powder, having a solubility in water of about 2 grams/ml at 25° C. It is used as a nasal decongestant, and is typically given in 60 mg doses about every three to four times daily. The resulting pelletized beads would be useful, for example, for producing a cough and cold pharmaceutical composition in solid dosage form.

Approximately 700 grams of a mixture of guaifenesin, dextromethorphan HBr, and pseudoephedrine HCl, on a moisture-free basis, were prepared in a weight ratio of approximately 83/4/13 (wlwiw), respectively. Three samples (Samples A through C) of pellets were prepared according to the following procedure, each sample varying in weight ratio of total active ingredients to binder. Each sample was prepared with the same, above defined mixture of the active ingredients. The binder for these samples was PEG 8000™, a polyethylene glycol available from Union Carbide Corporation.

A Robot-Coupe™ jacketed high-shear mixer with a 6-liter bowl was employed. The active ingredients were weighed out as described above and loaded into the bowl of the mixer, followed by approximately 15 seconds of dry mixing at an impeller speed of about 2000 rpm. The mixer was stopped, and the binder was placed into the bowl. Hot water, heated to approximately 73° C. was passed through the jacket of the mixer bowl. The thermal energy, along with the mechanical energy input by the impeller action, raised the temperature of the mixture inside the bowl to a temperature sufficient to melt the binder. At the end of each run, the pelletized beads were collected, weighed, and spread in thin layers to cool at room temperature. The pellets were screened, and fractions retained between a sieve #16 mesh and #80 mesh were retained for evaluation.

### EXAMPLE 2

### Test Methods

Test batches were prepared and the following test methods were utilized to characterize the resulting pellets.

A. Particle Morphology and Particle Size

The particle morphology of different samples was determined using polarized light microscopy (Olympus BH-2 or Leitz Sm-Lux-Pol polarized light microscope). Particle size distribution of the powdered materials was determined using sieve analysis technique. Sieve analysis was performed on granules using an ATM Sonic Sifter (ATM Corp., Milwaukee, Wis.) with a series of US standard sieves ranging in size from mesh #20 to #200. Approximately 10 grams of the test material was placed on the top sieve and vibrated for 5 minutes with an amplitude setting of 8. The fraction retained on each screen was determined and the average particle size assigned to each isolate was computed by average sieve sizes (in μm).

B. Powder Flow

The bulk density was determined by filling the powder sample into the tared graduated cylinder to the 100 mL mark. The graduated cylinder was weighed and the bulk density (Pb) calculated as ratio of the sample weight to sample volume. The graduated cylinder was then tapped using a VanKel Tap Densiometer Model 50-1200 (VanKel, Cary, N.C.). The tap density (ρₜ) was calculated as the ratio of the sample weight to the final sample volume after 1000 taps. No further volume reduction was observed after 1000 taps. The change in packing density as a result of the tapping procedure is expressed as the Carr Index (I) using the following equation:
I=[1−(ρ_{b}/ρₜ)×100

A value of Carr's Index≦25 is consistent with a good powder flow.

C. Active Ingredient Content of Pellets

The active ingredient content of the pellets was measured using HPLC technique. A 5 μm Waters Symmetry Shield RP18™ 150×4.6 mm column was used, along with a UV detector. The wavelength was set at 220 nm and the flow rate was set at 1.5 ml/min. The mobile phase consisted of 65 parts by volume sodium phosphate buffer and 35 parts by volume acetonitrile. A mobile phase consisting of 81% 0.005 M hexanesulfonic acid solution and 19% v/v acetonitrile was used for the assay of guaifenesin. If alternative active ingredients are utilized, they can be analyzed in a single run as well. The injection volume was 10 μl and the retention time of guaifenesin was 3.5 minutes, dextromethorphan HBr was 9.2 minutes, and pseudoephedrine HCl was 4.4 minutes. A reference standard was used for quantification of the amounts of drug present in each sample based on the average peak area of the standards.

### EXAMPLE 3

### Test Batch Analyzation

Four batches of each sample were prepared, and the product was mixed prior to testing. Multiple runs of each sample were made and each sample was tested after mixing. The pellets had the following properties shown in Table 1:

TABLE 1

| | ACTIVE | | | | MEDIAN |
|---|---|---|---|---|---|
| | INGREDIENT/ | BULK | TAP | | PAR- |
| | BINDER | DENSITY | DENSITY | CARR'S | TICLE |
| SAM- | WEIGHT | (ρ_{β}) | (ρ_{τ}) | INDEX | SIZE |
| PLE | RATIO | gm/cm³ | gm/cm³ | (I) | μm |
| A | 87.5/12.5 | 0.5076 | 0.5509 | 7.86 | 227.0 |
| B | 50/50 | 0.4648 | 0.5890 | 21.08 | 180.0 |
| C | 25/75 | 0.4735 | 0.6394 | 21.02 | 170.5 |

The pellets having the highest active ingredient content, Sample A, also exhibited the best flow characteristics.

### EXAMPLE 4

### Test Batch Analyzation

Several of the samples prepared were analyzed for active ingredient content according to the test methods outlined in Example 2. In Table 2, four (4) batches of Sample A, which were designed to contain 87.5 weight % total of active ingredients in each pellet with the remainder of the weight being the binder, were tested for active ingredient content on a moisture-free basis.

TABLE 2

| | | | | Dextro- | Pseudo- |
|---|---|---|---|---|---|
| | | | Guai- | methorphan | ephedrine |
| | | PAR- | fenesin | HBr | HCl |
| | | TICLE | Wt. % | Wt. % | Wt. % |
| SAM- | | SIZE | Theory/ | Theory/ | Theory/ |
| PLE | BATCH | μm | Actual | Actual | Actual |
| A | 1 | 150-850 | 72.91/72.04 | 3.65/3.76 | 10.94/10.9 |
| A | 2 | 150-850 | 72.91/73.96 | 3.65/3.76 | 10.94/10.94 |
| A | 3 | 150-850 | 72.91/73.94 | 3.65/3.56 | 10.94/10.76 |
| A | 4 | 150-850 | 72.91/73.45 | 3.65/3.81 | 10.94/10.87 |

The mean for guaifenesin was 73.35%, with a standard deviation of 0.90; the mean for dextromethorphan HBr was 3.72%, with a standard deviation of 0.11; and the mean for pseudoephedrine HCl was 10.87%, with a standard deviation of 0.08.

### EXAMPLE 5

### Test Batch Analyzation

Several of the samples prepared in Example 1 were analyzed for active ingredient content according to the test methods outlined in Example 2. In Table 3, three (3) samples taken from Batch 2 of Sample A, which were designed to contain 87.5 weight % total of active ingredients, were screened into various sieve fractions and tested for dextromethorphan HBr and pseudoephedrine HCl content on a dry weight basis.

TABLE 3

| | | | Dextromethorphan | Pseudoephedrine |
|---|---|---|---|---|
| | | PARTICLE | HBr | HCl |
| SAM- | | SIZE | Wt. % | Wt. % |
| PLE | BATCH | μm | Theory/Actual | Theory/Actual |
| A | 2 | 150-180 | 3.65/4.95 | 10.94/11.34 |
| A | 2 | 180-425 | 3.65/3.30 | 10.94/11.54 |
| A | 2 | 425-850 | 3.65/2.31 | 10.94/10.26 |

The mean for dextromethorphan HBr was 3.52%, with a standard deviation of 1.33; and the mean for pseudoephedrine HCl was 11.05%, with a standard deviation of 0.69.

### EXAMPLE 6

### Test Batch Analyzation

Sample C prepared above in Example 1 was tested for active ingredient content according to the test methods outlined in Example 2. The results are shown in Table 4 below:

TABLE 4

| | | | | Dextro- | Pseudo- |
|---|---|---|---|---|---|
| | | | Guai- | methorphan | ephedrin |
| | | PAR- | fenesin | HBr | HCl |
| | | TICLE | Wt. % | Wt. % | Wt. % |
| SAM- | | SIZE | Theory/ | Theory/ | Theory/ |
| PLE | BATCH | μm | Actual | Actual | Actual |
| C | 1 | 150-850 | 20.80/17.98 | 1.04/0.81 | 3.13/2.43 |

Sample C was designed to have a total active ingredient content of approximately 25 % by weight of each pellet.

### EXAMPLE 7

### Test Batch Analyzation

Sample D was prepared according to the procedure outlined in Example 1 except that Gelucire 50/13™ was used as the binder. This change in binder necessitated that the mixer bowl be heated to about 61° C. Also, the Gelucire 50/13™ was melted and then homogenized prior to addition to the mixer bowl. The pelletized beads had the following properties shown in Table 5:

TABLE 6

| | ACTIVE | | | | MEDIAN |
|---|---|---|---|---|---|
| | INGREDIENT/ | BULK | TAP | | PAR- |
| | BINDER | DENSITY | DENSITY | | TICLE |
| SAM- | WEIGHT | (ρ_{β}) | (ρ_{τ}) | CARR'S | SIZE |
| PLE | RATIO | gm/cm³ | gm/cm³ | INDEX (I) | μm |
| D | 87.5/12.5 | 0.4735 | 0.5956 | 20.49 | 243.5 |

It is to be understood that the embodiments and variations shown and described herein are merely illustrative of the principles of this invention and that various modifications may be implemented by those skilled in the art without departing from the scope and spirit of the invention.

What is claimed is:

1. A plurality of pellets for producing a solid dosage form of a pharmaceutical composition, comprising:
a plurality of active pharmaceutical ingredients; and
a binder,
wherein the pellets are formed by subjecting a mix of the plurality of active pharmaceutical ingredients and the binder to mechanical and thermal energy input in a mixing device until the binder has melted and the pellets have formed.

2. The pellets of claim 1, wherein the mixing device is a mixer.

3. The pellets of claim 2, wherein the mixer is a high speed or high shear mixer.

4. The pellets of claim 3, wherein the high shear or high-speed mixer is operated at about 2000 rpm.

5. The pellets of claim 1, further comprising cooling the pellets prior to producing the solid dosage form.

6. The pellets of claim 1, wherein the weight ratio of the plurality of active pharmaceutical ingredients to the binder is about 9:1.

7. The pellets of claim 1, wherein the weight ratio of the plurality of active pharmaceutical ingredients to the binder is about 3:1.

8. The pellets of claim 1, wherein the weight ratio of the plurality of active pharmaceutical ingredients to the binder is about 1:1.

9. The pellets of claim 1, wherein the weight % of the plurality of active pharmaceutical ingredients is about 25% to about 90%.

10. The pellets of claim 1, wherein the pellets exhibit a Carr's Index of less than about 25.

11. The pellets of claim 10, wherein the pellets exhibit a Carr's Index of less than about 10.

12. The pellets of claim 10, wherein the pellets exhibit a Carr's Index of between about 5 and 20.

13. The pellets of claim 1, wherein the binder has a melting point which is lower than the melting point of the active ingredient(s).

14. The pellets of claim 1, wherein the binder is capable of forming liquid adhesion for particle agglomeration.

15. The pellets of claim 1, further comprising at least one inactive ingredient.

16. The pellets of claim 15, wherein the at least one inactive ingredient comprises microcrystalline cellulose, croscarmellose sodium, methacrylic acid, vegetable stearic acid, vegetable cellulose, titanium dioxide, silica, glyceryl triacetate, silica, vegetable magnesium stearate, vegetable glycerin, or riboflavin, or a mixture thereof.

17. The pellets of claim 15, wherein the at least one inactive ingredient comprises a lubricant, diluent, compression agent, dispersion agent, disintegrating agent, coloring agent, flavoring agent, preservative, or a combination thereof.

18. The pellets of claim 1, wherein the solid dosage form comprises a tablet, capsule, pill, granule, powder, or a combination thereof.

19. The pellets of claim 1, wherein the active pharmaceutical ingredients are present in an amount effective to treat a cough or cold.

20. The pellets of claim 19, wherein the active pharmaceutical ingredients comprise an expectorant, a cough suppressant, and a nasal decongestant.

21. The pellets of claim 20, wherein the expectorant comprises guaifenesin, the cough suppressant comprises dextromethorphan hydrobromide, and the nasal decongestant comprises pseudoephedrine hydrochloride.

22. The pellets of claim 20, wherein the weight ratio of the expectorant, cough suppressant, and nasal decongestant is about 83:4:13 on a dry weight basis.

23. The pellets of claim 20, further comprising an analgesic.

24. The pellets of claim 23, wherein the analgesic comprises aspirin, ibuprofen, or acetaminophen, or a combination thereof.

25. The pellets of claim 1, wherein the active pharmaceutical ingredients are present in an amount effective to treat an allergy, cold or sinus or any combination thereof.

26. A composition comprising a method of treating a recipient compromising administering to the recipient the pellets of claim 1.

27. A method of preparing pellets including multiple active pharmaceutical ingredients, the method comprising the steps of:
a) adding multiple active pharmaceutical ingredients to a mixing device;
b) adding at least a binder to the mixing device;
c) adding thermal energy to the mixing device; and
d) mixing the multiple active ingredients and the binder until pellets are formed.

28. The method of claim 27, further comprising cooling the pellets.

29. The method of claim 27, wherein the steps a)-d) are conducted in the order set forth in claim 27.

30. The method of claim 29, further comprising a step e) cooling the pellets, conducted after the step d).

31. The method of claim 27, wherein the mixing device is a mixer.

32. The method of claim 31, wherein the mixer is a high speed or shear mixer.

33. The method of claim 27, wherein the mixing device is operated from about 1500 to about 2000 rpm.

34. The method of claim 32, wherein the high speed or high shear mixer is jacketed.

35. The method of claim 34, wherein the thermal energy is added to the high shear or high speed mixer by passing heated liquid or gases through the jacket.

36. The method of claim 27, wherein the pellets are from about 150 μm to about 250 μm average diameter.

37. The method of claim 35 further comprising cooling the pellets.

38. The method of claim 27, wherein the solid dosage form pharmaceutical composition comprises tablets, capsules, pills, granules, or powder.

39. A method of treating a cold and cough, the method comprising:
adding an expectorant, a cough suppressant, and a nasal decongestant to a mixing device;
adding at least a binder to the mixing device;
adding thermal energy to the mixing device;
mixing the expectorant, cough suppressant, and nasal decongestant and binder until pellets are formed;
forming a solid dosage form of a pharmaceutical composition comprising the pellets; and
administering the pharmaceutical composition to a recipient.

40. The method of claim 43, further comprising cooling the pellets.

41. The method of claim 43, wherein the mixing device comprises a mixer.

42. The method of claim 41, wherein the mixer is a high shear or high speed mixer.

43. The method of claim 42, wherein the mixer is jacketed.

44. The method of claim 39, wherein the recipient is a human.

45. The method of claim 39, wherein the solid dosage form comprises tablets, capsules, pills, granules, powder, or a combination thereof.

46. The method of claim 39, wherein the expectorant comprises guaifenesin.

47. The method of claim 46, wherein about 200 to about 400 mg guaifenesin is administered to the recipient every four hours, per day.

48. The method of claim 39, wherein the cough suppressant comprises dextromethorphan hydrobromide.

49. The method of claim 48, wherein about 10 to about 20 mg dextromethorphan hydrobromide is administered to the recipient every four hours, per day.

50. The method of claim 39, wherein the nasal decongestant comprises pseudoephedrine hydrochloride.

51. The method of claim 50, wherein about 60 mg pseudoephedrine hydrochloride is administered to the recipient every four hours, per day.
